(19) United States
(12) Patent Application Publication
(10) Pub. No.: US 2012/0010160 A1
(43) Pub. Date: Jan. 12, 2012

(54) Rapidly Dissolving Oral Strip of Sugar for Hypoglycemia

(76) Inventor: Tania Degian, Toronto (CA)

(21) Appl. No.: 13/088,567

(22) Filed: Apr. 18, 2011

Related U.S. Application Data
(60) Provisional application No. 61/361,547, filed on Jul. 6, 2010.

(51) Int. Cl. A61K 31/7004; A61B 19/02; A61P 3/08

(52) U.S. Cl. 514/23; 206/438

(57) ABSTRACT

A method of orally administering measured doses of sugar into the bloodstream for controlling glucose levels, particularly for those individuals with diabetes or those who experience difficulty maintaining healthy blood sugar levels. The method comprises providing packets of orally ingestible strips of a disintegrating matrix containing a dose of sugar to supplement food intake between meals or to rapidly increase glucose levels in an emergency situation. Hypoglycemia is a condition of low blood sugar (glucose) levels in the bloodstream, which can have varying reactions on individuals ranging from mild to severe. The disclosed matrix and method rapidly introduces sugar into the blood stream for combating a hypoglycemic reaction. The strips are easily deployable and quickly dissolve once placed on the tongue and mixed with saliva. They can be packaged in a small, easy-to-dispense, and transportable case that allows rapid use when a sugar supplement is required.

### CROSS REFERENCE TO RELATED APPLICATION

This application claims the benefit of U.S. Provisional Application No. 61/361,547 filed on Jul. 6, 2010, entitled “Sugaraid.”

### BACKGROUND OF THE INVENTION

1. Field of the Invention

The present invention relates to hypoglycemic countermeasures. More specifically, the present invention pertains to a method of dispensing orally ingestible and dissolvable sugar strips that contain a dosage of glucose monosaccharide sugar to increase an individual's glucose levels.

Hypoglycemia is a condition of low blood sugar (glucose) in the bloodstream, which can manifest in varying symptoms and cause serious health problems for those with diabetes. The condition may occur when an individual's body metabolizes blood sugar too quickly, if blood sugar is released too slowly, or if too much insulin is introduced into the bloodstream. Insulin is a naturally occurring hormone that reduces blood sugar, produced by the pancreas in response to elevated glucose levels in the blood. For those with diabetes, insulin production is reduced to the point that insulin injections or other medication may be required to correct the blood sugar levels. If a diabetes patient injects too much insulin or intakes too much diabetes medicine, if they do not eat properly, or if an individual increases the rate of glucose consumption through exercise or activity without increasing food intake, that individual may experience a hypoglycemic reaction.

Symptoms of hypoglycemia range from mild to severe depending on the individual and if the individual has a specific condition. Mild reactions include irritability, blurry vision, general discomfort and the like, while more serious reactions may include fainting, seizures or coma.

The present invention is a method of orally introducing a dosage of sugar that is rapidly absorbed into the bloodstream to combat the signs and symptoms of low glucose levels. The sugar is introduced via a strip of solid matrix that is dissolvable when introduced onto the tongue and mixed with saliva. The dissolvable strip supplements food intake for short periods of time between meals or in emergency situations where a diabetic individual requires sugar to counteract a hypoglycemic reaction.

2. Description of the Prior Art

The application of the sugar in a dissolvable matrix form is desirable over solid forms and tablet forms of sugar delivery that require longer periods of time to metabolize and introduce into the bloodstream. Several patents have been described in the art for dissolving film strips for oral use, including those for oral hygiene or to introduce a medication into the user. While these patents may have sugar as an ingredient, their composition is not a pure sugar compound and their intended uses differ from specifically elevating glucose levels in the bloodstream in response to or anticipation of hypoglycemia.

U.S. Pat. No. 6,284,264 to Zerbe describes one such device in which therapeutic agents or breath freshening agents may be introduced into the oral cavity via a water-soluble polymer. The polymer is in film form, which actively disintegrates in the user's mouth. U.S. Pat. No. 5,629,003 to Horstmann is another such device in which a film of gel former that is utilized as a carrier to introduce a substance into an individual through oral ingestion. Finally, U.S. Pat. No. 7,678,397 to MacQuarrie and U.S. Pat. No. 6,596,298 to Leung describe edible films for combating oral hygiene concerns and for delivering medical compounds to the user.

The prior art patents lack a method of providing a dissolvable film matrix that introduces a known quantity of sugar into the user's bloodstream. Controlling blood sugar is a problem for diabetics and those with fast metabolisms. For diabetics, an introduction of an uncontrolled level of sugar may cause an unwanted spike in glucose levels, which is not desired. Glucose levels must stay within a healthy range. If a diabetic is hypoglycemic and introduces an unmeasured amount of sugar into their body, the spike in glucose levels will have a negative effect. The glucose levels will spike above acceptable ranges, necessitating further action such as insulin injections or medication.

Therefore, the primary function of the disclosed invention is to disclose a method of introducing controlled levels of sugar for individuals with blood sugar problems. The strips contain a known quantity of sugar, which is quickly introduced into the bloodstream in a controlled manner, as opposed to candy snacks or food with high levels of sugar. Alternative means of sugar introduction may also take long to metabolize in the body, which can be a critical factor in an emergency situation.

### SUMMARY OF THE INVENTION

In view of the foregoing disadvantages inherent in the known types of dissolvable film strips now present in the prior art, the present invention provides a new method of sugar delivery which utilizes a film strip with a quantity of sugar, wherein the same can be utilized for providing convenience for the user when controlling glucose levels in the bloodstream and combating hypoglycemia.

It is therefore an object of the present invention to provide a method of sugar introduction into individuals, particularly diabetics, wherein a film strip is provided that quickly dissolves in the presence of saliva, and is quickly introduced into the bloodstream.

Another object of the present invention is to provide a method of controlling blood sugar between meals and in situations where food is unavailable for maintaining an acceptable level of glucose in the bloodstream.

Other objects, features and advantages of the present invention will become apparent from the following detailed description taken in conjunction with the accompanying drawings.

### DETAILED DESCRIPTION OF THE INVENTION

The present invention contemplates utilizing a dissolvable polymer matrix material that is ingestible, and one that contains a measured dosage of sugar. The strips may be contained in a transportable container for travel-use, and markings on the container may show the dosage of sugar, in grams per strip. The present invention is a method of use that involves providing the strips to diabetics and those with blood sugar maintenance problems.

Between meals and in situations where food is not readily available, diabetics have to be aware of their blood sugar levels. This level must be carefully monitored in these individuals, and must fall within an acceptable range of glucose levels, as measured by a blood sampling device. After a period without food, or after physical exercise, glucose levels in the blood may be low. This can lead to symptoms of irritability to fainting, depending on the severity of the glucose level and the condition of the individual. In these situations, ingestion of food or sugar supplement is required to avoid complications.

First, an individual may carry or transport the strips in a personal container, which distributes individual strips among a plurality of strips. The strips contain several grams of sugar, in the form of glucose that a patient may utilize to elevate his or her blood sugar levels. Knowing the dosage avoids overcompensating and ingesting too much sugar than is necessary to correct the deficiency in blood sugar in the body.

The sugar strips are introduced into the oral cavity of the user, either buccally (along the cheek), sublingually (below the tongue), or on the tongue itself. Once mixed with saliva, the film rapidly dissolves and enters the bloodstream directly from the mouth, without the need for metabolism in the stomach and small intestine. This form of transmucosal sugar delivery is superior when time is critical.

The film itself is a combination of hydrophilic polymers and a dosage of pure dextrose, which is a naturally occurring type of glucose. Glucose is a metabolized form or carbohydrate sugar, such a sucrose, fructose and lactose, and is a simple sugar utilized by the present invention to rapidly increase blood sugar levels in the body. The amount of strips required by the individual will be based on the blood sugar level at the time. The use of a controlled dose allows the user to know how many strips to ingest to raise the blood sugar levels to an acceptable range, avoiding overshoot and further complications.

In total the disclosed method is an advancement in the art by providing a means to controllably elevate blood sugar levels in the body, and to prevent complications with hypoglycemia. Diabetics and those that quickly metabolize blood sugar will benefit from having a method of increasing those levels in an easy-to-use and easy-to-dispense strip of polymer matrix/sugar blend. The strips provide a means to correct blood sugar levels rapidly, without the need to find a food source or when situations arise where there is no source of food.

With respect to the above description then, it is to be realized that the optimum relationships of the invention, to include variations in size, materials, shape, form, function and manner of operation, assembly and use, are deemed readily apparent and obvious to one skilled in the art, and all equivalent relationships to those described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact operation shown and described, and accordingly, all suitable modifications and equivalents may be resorted to, falling within the scope of the invention.

I claim:

1) A method of elevating glucose levels in the blood stream, comprising the steps of:
measuring a sample of blood to determine glucose levels in one's blood stream utilizing a glucose testing device,
determining a degree of blood sugar deprivation in said measured sample,
dispensing a quantity of strips of dissolvable polymer matrix and sugar supplement from a dispensing container,
placing said quantity of strips in an oral cavity, one at a time, for rapid absorption of sugar into the blood stream.
waiting several minutes and remeasuring a sample of blood to monitor a change in glucose levels after application of said strips.

2) A film matrix for elevating glucose levels in the blood stream, comprising:
a strip of dissolvable polymer matrix and dextrose,
said strip is contained within a dispensing container with a plurality of other similar strips for easy dispensing.
